(19) United States
(12) Patent Application Publication
(10) Pub. No.: US 2014/0066893 A1
(43) Pub. Date: Mar. 6, 2014

(54) Roller Clamp

(71) Applicant: Marco Valentini, St-Sulpice (CH)

(72) Inventor: Marco Valentini, St-Sulpice (CH)

(73) Assignee: CARE-FUSION CORPORATION, San Diego, CA (US)

(21) Appl. No.: 13/988,343

(22) PCT Filed: Nov. 19, 2010
(86) PCT No.: PCT/EP10/67854

§ 371 Date: Aug. 5, 2013

(51) Int. Cl. A61M 39/28; A61M 5/168

(52) U.S. Cl. CPC A61M 39/28; A61M 5/16813
USPC 604/506; 251/6; 604/250

(57) ABSTRACT

A roller clamp includes at least two opposing and spaced apart side walls, a guide wall, a further wall opposite the guide wall defining an aperture therethrough, a hollow body and a roller. The hollow body has a body aperture therethrough. The roller comprises a roller wheel having two axial projections protruding from a center of each side of the roller. Each side wall is provided with an opposing guide groove on the interior surface thereof. The two axial projections of the roller wheel are slidingly seated in the guide grooves so that the roller can move in the aperture of the further wall as the projections slide in the grooves. At least one respective long edge of each corresponding guide groove is provided with at least one notch. The resulting at least one pair of corresponding notches can at least partially seat the two projections.

### CROSS REFERENCE TO RELATED APPLICATIONS

This application claims the priority benefit of PCT International Patent Application Serial No. PCT/EP2010/067854, the entire disclosure of which is hereby expressly incorporated by reference herein.

### TECHNICAL FIELD

This application relates generally to a roller clamp for use in gravity infusion.

### BACKGROUND

The present disclosure relates to a roller clamp and in particular to a roller clamp for use in gravity infusion. The roller clamp regulates the flow rate of a medical fluid (for example a solution of a drug to be administered to a patient, or blood) flowing through a tube.

Gravity infusion means the infusion of a medical fluid to a patient under the force of gravity, i.e., as opposed to infusion carried out using an infusion pump. Typically, a standard infusion set is used to infuse the fluid. An example of a standard infusion set is shown in FIG. 1.

The infusion set includes a piercing spike 20 which may either be a sharp spike for piercing rubber stoppers or rounded and blunt for insertion into a bag. The spike contains one channel for fluid and optionally a second channel for venting. A vent 21 is usually present in the vicinity of the piercing spike to allow air to flow into the drop chamber 22 (described below). The vent may be provided with a bacterial filter to prevent bacteria from entering the equipment.

A drop chamber 22 is provided and has a drop generator 23 at the top of the drop chamber which produces drops of a certain size. Drops from the drop generator fall into the drop chamber such that the chamber is partially filled with liquid. This prevents air bubbles from entering the tubing which would be harmful to a patient. A particle filter may be provided at the lower aperture of the drop chamber.

A connection tubing 24 connects the drop chamber 22 with the patient. The connection tubing is usually around 150 cm long and can be manufactured from PVC. The tubing is shown shortened in FIG. 1 for clarity. The connection tubing 24 typically has a standardised diameter.

At the end of the connection tubing is a Luer fitting 25 which is standardised for connection to all other pieces of apparatus having a standard Luer cone. The person skilled in the art will appreciate that the Luer fitting 25 can be fitted to a hypodermic needle for infusing the medical fluid into the circulatory system of a patient, for example into a vein.

Between the drop chamber 22 and the Luer fitting 25 and engaging with the connection tubing is a roller clamp 26. The present disclosure is concerned with an improved roller clamp but a standard roller clamp as known in the art 26 will now be described for background information.

A prior art roller clamp 26, illustrated in FIGS. 2 and 3 comprises two opposing side walls 27 having a pair of guide grooves 30 that are aligned with each other and face each other. A flow-regulating roller 28 is provided having axially-projecting shafts 29 protruding from the centers of each side of the roller. The roller is omitted from FIG. 2 but is shown in outline in FIG. 3, for clarity. The shafts of the roller are captured by and seated in the guide grooves 30 so that the roller can move up and down the guide grooves as indicated by arrows in FIG. 3.

The entire roller clamp, comprising four side walls as illustrated in FIG. 1, in an open-ended boxlike construction, is dimensioned and configured to receive the connector tubing 24. In use the tubing passes through the roller clamp, between the two opposing side walls 27, the roller 28 and a guide wall 31 which is opposed to the roller.

In a prior art roller clamp 26, the surface of the guide wall 31 converges along its length toward the position of the guide grooves 30 in the downward direction of the guide grooves i.e., in the direction of the arrows in FIG. 3. This tends to urge the connector tubing within the roller clamp toward the guide grooves and thus toward roller 28.

Thus, rolling the roller downwardly along the guide grooves in the direction of the gradually closer guide wall 31 (direction of the arrows in FIG. 3) causes the roller to impinge against the connector tubing. As the roller impinges on the tubing, the tubing becomes squeezed, as it is a flexible material such as PVC and the lumen of the infusion tubing therefore becomes smaller. In this way, by narrowing of the lumen, the flow rate of liquid passing through the connector tube can be regulated.

One problem with prior art of roller clamps is that, over time the flexible connector tubing material gives way as it loses its mechanical strength under compression and the diameter of the tubing lumen decreases, resulting in a corresponding decrease of the flow rate. In this instance, the roller clamp would need to be readjusted at a different point along the tubing to achieve the originally set rate of infusion.

Moreover, if the tubing material gives way, then the roller clamp is likely to shift in position, and again would need to be repositioned to re-establish the correct flow rate.

Furthermore, when moving a patient or moving the infusion apparatus, the roller clamp can shift out of position quite easily, again resulting in an incorrect rate of infusion.

The present disclosure aims to overcome these problems.

### SUMMARY

In accordance with one embodiment, a roller clamp comprises at least two opposing and spaced apart side walls, a guide wall, a further wall opposite the guide wall defining an aperture therethrough, a hollow body and a roller. The hollow body has a body aperture therethrough. The body aperture is dimensioned and configured to house a resilient connection tube of a gravity infusion set therethrough. The hollow body is defined by the at least two opposing and spaced apart side walls. The guide wall and the further wall are opposite the guide wall. The roller comprises a roller wheel having two axial projections protruding from a center of each side of the roller. The roller wheel protrudes through the aperture of the further wall. The spacing between the guide wall and the further wall decreases over the length of the guide wall and the further wall. Each side wall is provided with an opposing guide groove on the interior surface thereof. The two axial projections of the roller wheel are slidingly seated in the guide grooves so that the roller can move in the aperture of the further wall as the projections slide in the grooves. At least one respective long edge of each corresponding guide groove is provided with at least one notch. The resulting at least one pair of corresponding notches can at least partially seat the two projections.

In accordance with another embodiment, a method of regulating fluid infusion through a connection tube is provided. The method comprises inserting the tube through the body aperture of a roller clamp. The method further comprises rolling the roller downwardly in the direction of greater to lesser spacing between the guide wall and the further wall, until the axial projections are seated in notches whereby the tube diameter has been constricted to a desired extent to provide a desired flow rate of fluid through the tube.

### BRIEF DESCRIPTION OF THE DRAWINGS

It is believed that certain embodiments will be better understood from the following description taken in conjunction with the accompanying drawings in which:

FIG. 1 is a schematic illustration of a gravity infusion set;

FIG. 2 is a cross-sectional view of a roller clamp as known in the prior art;

FIG. 3 is a further cross-sectional view of a prior art roller clamp;

FIG. 4 is a perspective view of a roller clamp according to the invention;

FIG. 5 is a cross-sectional view of a roller clamp according to the present disclosure;

FIG. 6 is a plan view of a roller clamp according to the present disclosure;

FIG. 7 is a side view of a roller clamp according to the present disclosure;

FIGS. 8 and 9 are corresponding cross-sectional views of a roller clamp according to the present disclosure; and

FIGS. 10 and 11 are side views of two roller clamps according to the present disclosure.

### DETAILED DESCRIPTION

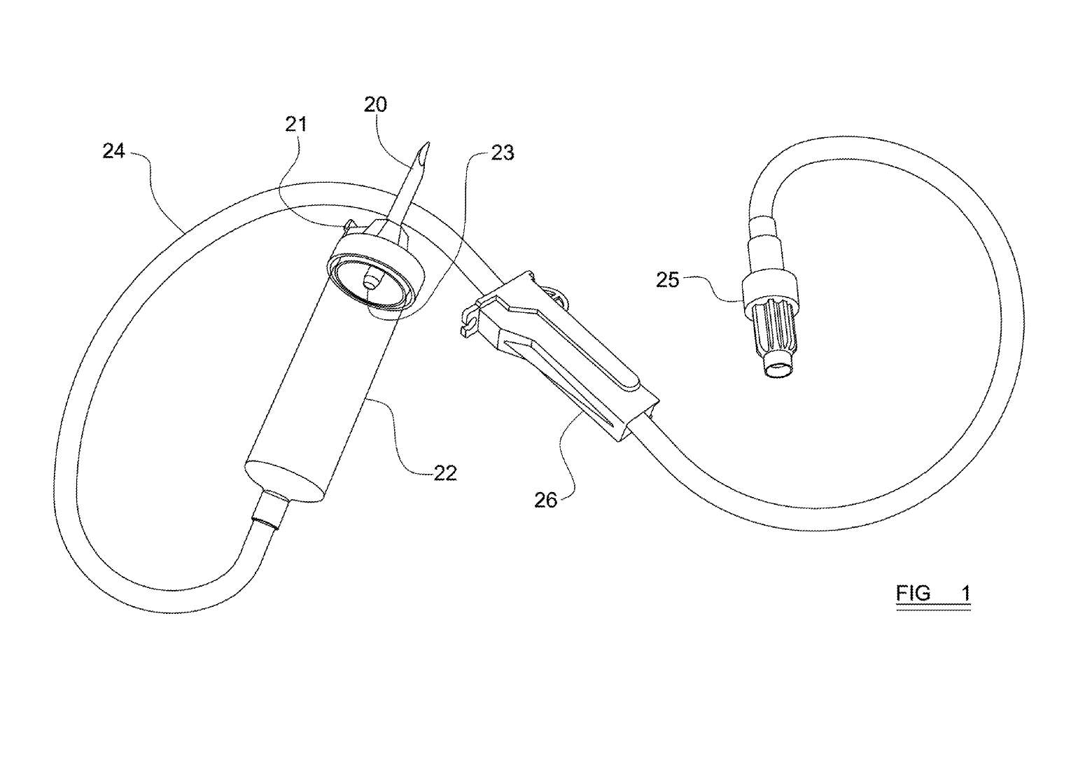

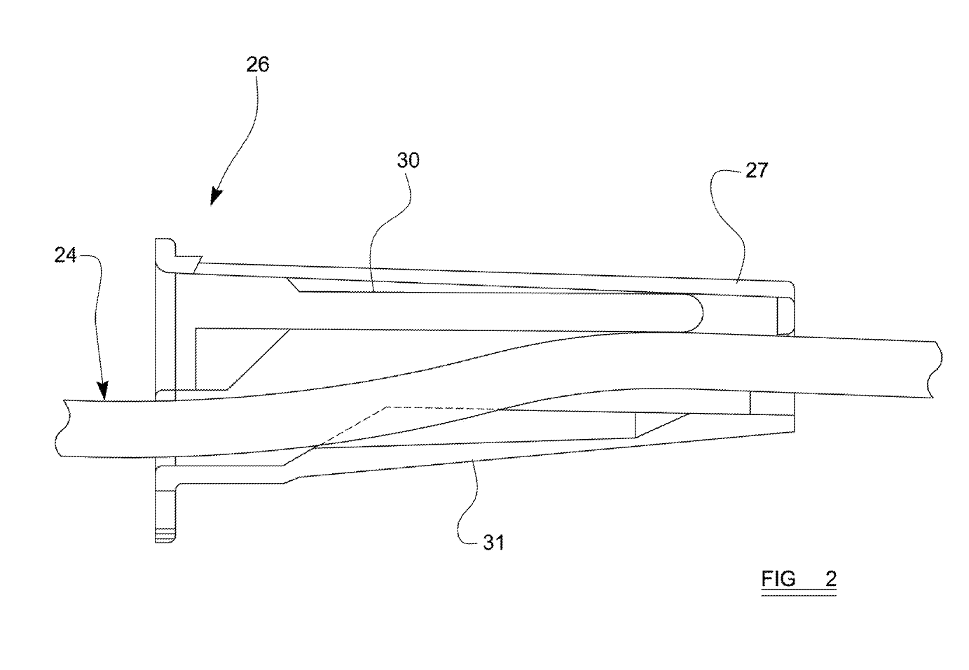

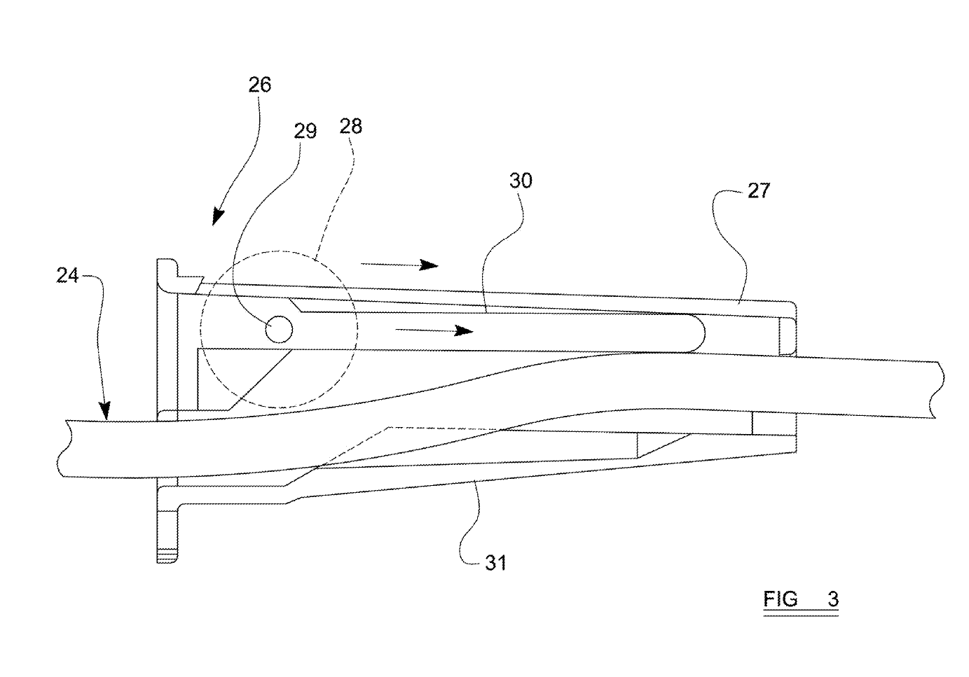

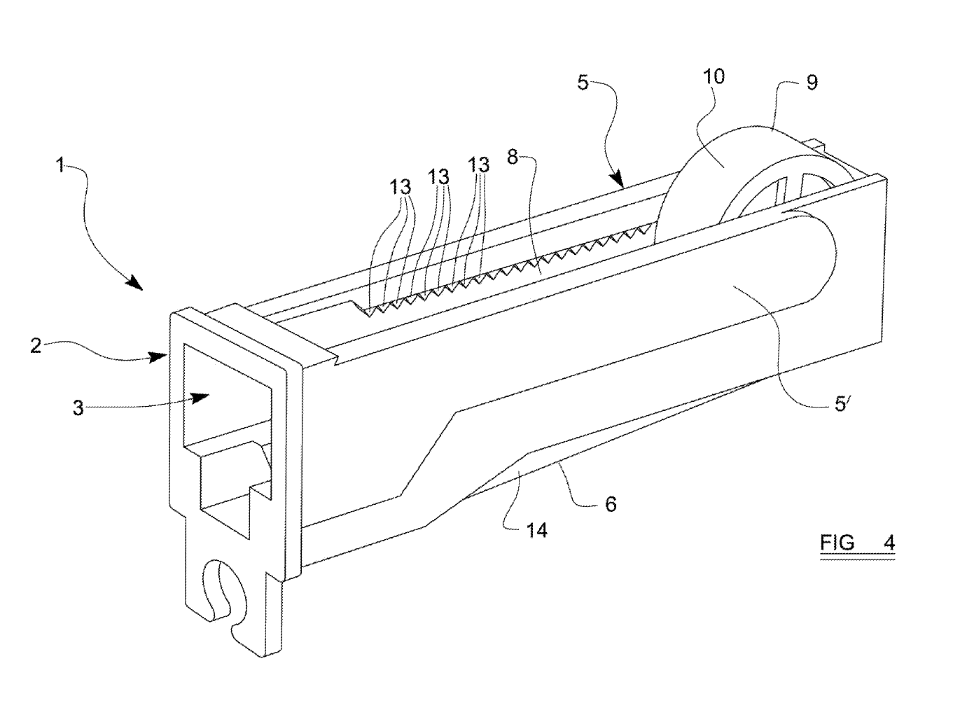

Embodiments are hereinafter described in detail in connection with the views and examples of FIGS. 1-42, wherein like numbers indicate the same or corresponding elements throughout the views. According to one embodiment, as illustrated in FIGS. 1 and 2, a hand-held pneumatic oscillating tool 30 (hereinafter “oscillating tool”) is provided that can include a housing 31 and can extend between a front end 32 and a rear end 34. As illustrated in FIG. 2, an air inlet 36 can be disposed at the rear end 34 and supported by the housing 31 and can include a base portion 35 and a stem portion 37. The base portion 35 of the air inlet 36 can be coupled with an air compressor (not shown) or another source of pressurized air or other fluid. The pressurized air can facilitate selective powering of the oscillating tool 30 which can actuate an accessory blade 38 such that the oscillating tool is operable as a hand-held cutting tool.

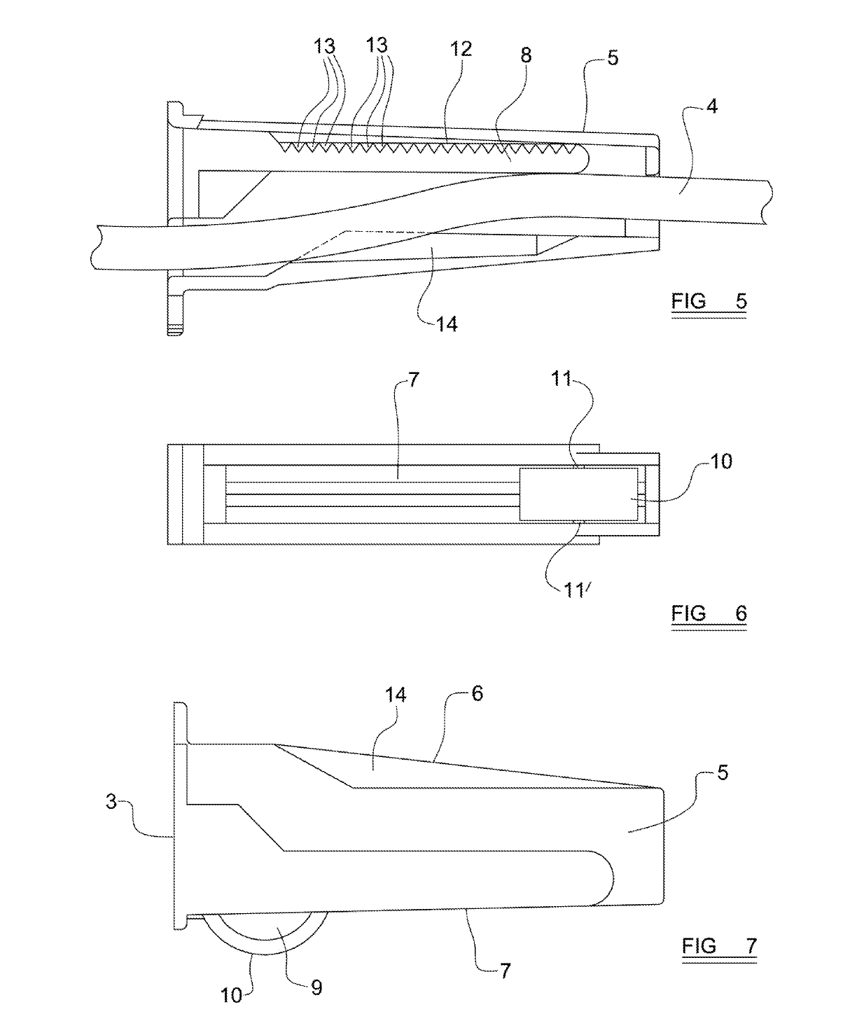

As will be seen by comparing FIGS. 2 and 5 in particular, the roller clamp 1 can be a generally elongate hollow boxlike structure 2 having two open ends forming an aperture 3 through which a connector tube 4 can pass. The roller clamp therefore has four generally rectangular shaped sides 5, 5′, 6 and 7.

Each of the two side walls 5, 5′ which face each other are each provided with an opposing guide groove 8. Of the other two sides of the clamp, one side 7 can be open, in the form of a frame to receive a roller, whereas its opposing side 8 can be a guide wall having a channel 14 defined therein. The channel can be tapered from the upper part of the roller clamp to the lower part of the roller clamp. That is to say that the depth of the channel decreases in the direction of top to bottom in the roller clamp.

The roller clamp 1 also has a region of increased cross sectional area at the top of the roller clamp such that the top end opening of the roller clamp can be larger than the bottom end opening. The channel 14 can preferably be a V-configuration which tapers as described above from approximately the shape of an equilateral triangle at the top opening of the roller clamp to no channel at all, i.e., flush with the guide wall 6 of the roller clamp 1 at the bottom end opening.

The two opposed guide grooves 8 house therebetween the roller 9. The roller comprises a wheel 10 having two centrally-protruding axial projections 11, 11′. The axial projections 11, 11′ project from the center of the wheel 10 and are housed in the grooves 8. Therefore, it is to be appreciated that the roller 9 can move up and down the roller clamp 1 by way of the projections 11, 11′ moving in the grooves 8. The roller 9 can be operated by an operator's finger or thumb. Preferably, the surface of the wheel 10 can be serrated to provide better traction on an operator's finger.

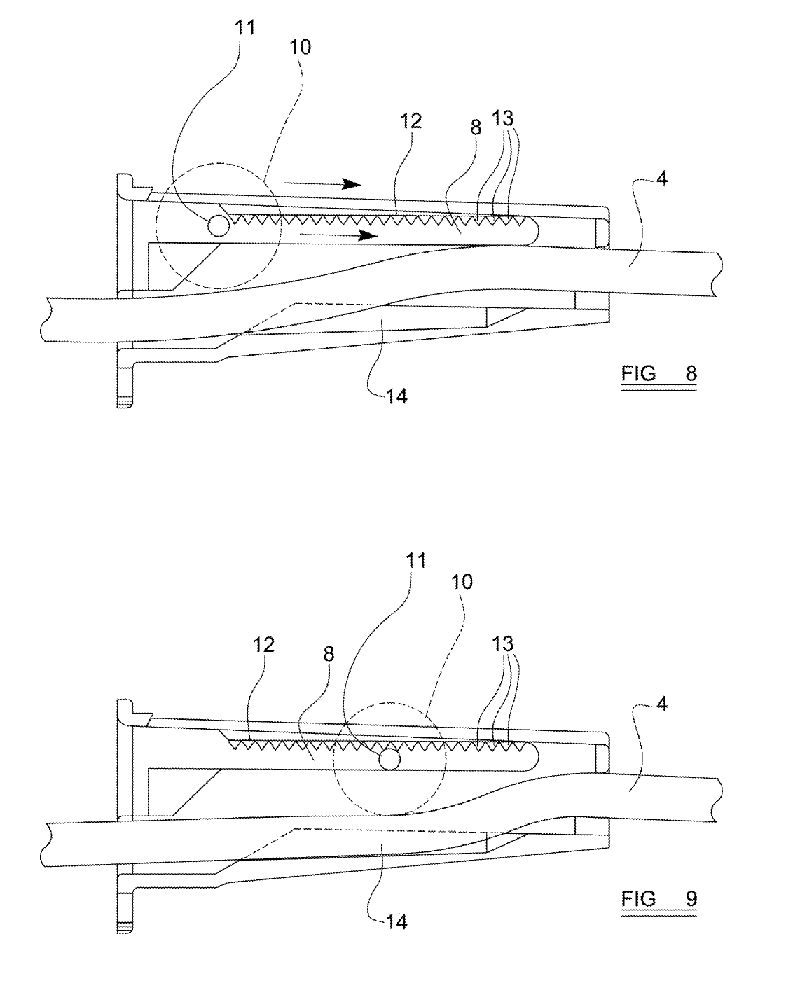

In use, the wheel 10 can be pushed up the grooves 8 to the top of the roller clamp and the connection tubing 4 can be inserted in the top of the roller clamp 1 which has a larger opening than the bottom of the roller clamp. The top end opening of the roller clamp can be of sufficient cross-sectional area to accommodate both the connection tubing 4 and the roller 9, when the roller 9 can be positioned at the top of the guide grooves 8. The connection tube then passes through the roller clamp and out of the bottom, smaller opening. Thus in this position as illustrated in FIG. 8 (with the wheel 10 in outline), there can be sufficient space in the larger open end of the roller clamp 1 to house both the tubing 4 and the roller wheel 10, without the wheel impinging on the connection tubing 4.

However, as discussed above, the bottom opening of the roller clamp has a smaller cross sectional area than the opening at the top and, moreover, the channel 14 in the guide wall 6 tapers until flush with the guide wall. Therefore, moving in the direction from top to bottom in the roller clamp 1, in the direction of the arrows from FIG. 8, there can be progressively less space to accommodate both the roller wheel 10 and the connection tubing 4.

Accordingly, when an operator moves the roller wheel 10 downwardly (i.e., in the direction of the arrows in FIG. 8) by manipulating it with his or her finger or thumb, the roller wheel 10 impinges progressively more on the connection tubing 4. Therefore, as the roller wheel 10 is pushed in a downward direction, the connection tubing 4 (which can be a flexible material such as PVC) becomes squeezed to an increasingly greater extent. Squeezing of the tube causes the lumen therethrough to be reduced in diameter and therefore, when a medical fluid is being infused via the tubing, the flow rate will be decreased as the roller wheel is moved downwardly and the tubing decreases in diameter. This situation is illustrated in FIG. 9, again with roller wheel 10 in outline.

The wheel 10 can lie in the notches 13 formed in the sides of the opposed guide grooves 8 that are closest to the exterior surface of the roller clamp 1.

As will be appreciated from the above discussion, the axial projections 11, 11′ on the roller 9 which are seated in the facing guide grooves 8 will therefore be seated in the notches 13. As will also be appreciated, as the connector tube 4 can be a resilient material, as the roller wheel 10 squeezes against the tube 4, the tube 4 will exert an opposing force on the wheel 10 which will urge the projections 13 into the gaps between the notches, where the projections will remain seated.

Accordingly, when a user rolls the roller wheel 10 in a downward direction in the roller clamp 1, the projections 11 will enter and exit successive gaps between notches 13 in a number of defined graduated steps defined by the notches. Once the user has chosen the desired rate of infusion, the projections 11 will remain seated in the chosen position.

This arrangement, as opposed to having no notches and a continuously variable range of movement for the projections will mean that it is much more unlikely for the projections 11 to become unseated from the notches 13 and therefore for the wheel 10 to move out of position. This substantially avoids the aforementioned problem whereby, for example when a patient moves, the wheel inadvertently moves to an undesired location.

As illustrated, the notches 13 can be formed in just one edge 12 of each corresponding guide groove 8; this can be either the edge 12 closest to the exterior of the roller clamp 1, as illustrated, or the edge closest to the connection tubing, in use. Alternatively, the notches can be provided in both edges or in a region of each edge or both edges.

In the event that the connector tubing 4 collapses, i.e., loses a certain amount of its structural integrity or resilience, the projections 11 of the roller wheel will nevertheless not fall out of their seated position and therefore the roller wheel will not drop axially downwardly thereby affecting the rate of flow.

Whilst the notches 13 are depicted as being a series of triangular points, it is to be appreciated that the notches could also have a smooth profile, i.e., bumps rather than points. Moreover, whilst the notches are depicted as being a continuous series of notches, like a serrated knife, it is to be appreciated that there could just be one notch, or two or more notches separated by a non-notched region of the grooves. This would allow the wheel to be “set” in particular pre-defined locations in the grooves.

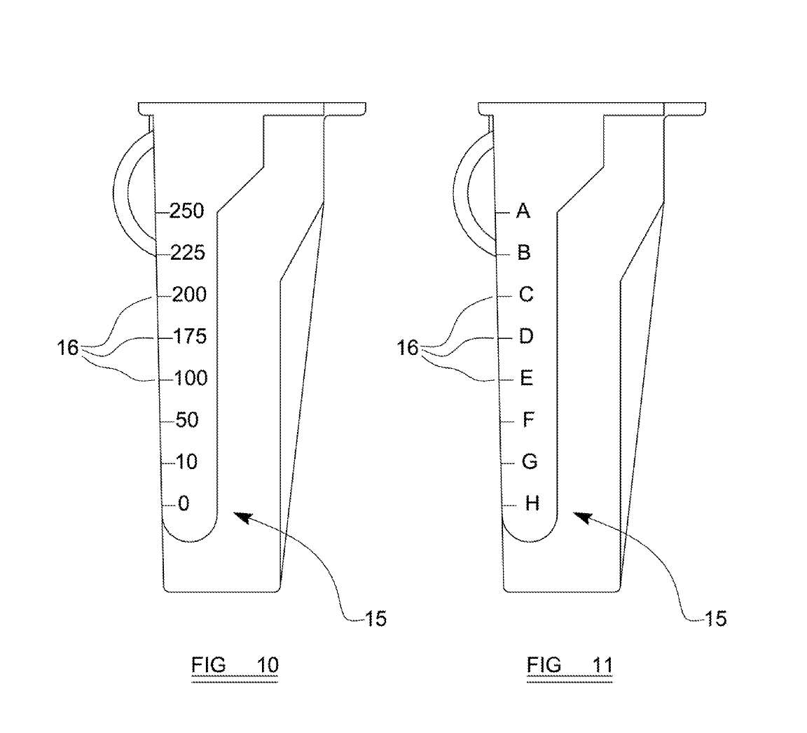

In addition, as illustrated in FIGS. 10 and 11 the roller clamp 1 of the present disclosure can preferably be provided with a visual scale 15 for the operator to give an indication of the infusion rate provided by a particular roller wheel position.

As explained above, the position of the wheel in the long axis of the roller clamp 1 provides a different level of compression on the infusion tube and therefore a different rate of infusion depending on the amount of constriction of the lumen.

As also explained further above, an infusion set typically contains a drop chamber including a drop generator. This is often standardised such that a number of drops can be equivalent to a particular volume of liquid (for example 20 drops is 1 ml). An operator can determine an approximate rate of infusion by counting the number of drops issuing from the drop generator to yield an infusion rate, for example, 100 ml per hour.

Because the items in an infusion kit are standardised (e.g. drop generator and tube diameter), a guidance scale 15 can be printed or otherwise provided on the side of the roller clamp 1 because the effect on the infusion rate by operating the roller clamp 1 can be predictable. For example, in a particular infusion set, where the roller wheel is at the close to the top of the roller so that it begins to impinge on the tubing, it might be known that the infusion rate is approximately 250 ml per hour. Moving the roller wheel downwardly along a certain number of notches would be known to reduce the infusion rate by half i.e., to 125 ml per hour. When the roller wheel is almost, but not fully, in the lowermost position, the flow can be restricted to such an extent that the infusion rate can be 5 ml per hour. This provides a useful guide to an operator as to the approximate effect on infusion rate of operating the roller wheel.

Of course, gravity infusion is inherently an inaccurate technique, although it can be extremely useful and widespread in clinical practice. Whilst the scale on the side of the roller clamp 1 is intended to provide useful information to an operator, it is not intended as a replacement to the traditional method of counting drops to determine actual flow rate and so, rather, the scale is an enhancement to the roller clamp 1 providing additional useful information. The operator can readily see, from the scale on the roller clamp 1, the approximate position that the wheel needs to be in order to provide a particular infusion rate.

In addition to a scale 15 on one side of the roller clamp 1 giving an indication of an infusion rate in millilitres per hour, or another suitable scale, there could be a second scale on the same side of the roller clamp 1 or preferably on the opposing side of the roller clamp 1 which is solely intended to identify the location of the roller wheel once the wheel has been set in the desired position. The scale could, for example, be a series of numbers (e.g. 1-10) or even a series of letters. Alternatively, the positioning scale could simply be a series of identifiers or marks such as horizontal lines.

Therefore, once the operator has set the desired infusion rate by drop counting, the operator could also make a note of the indicia 16 provided by the positioning scale on the side of the roller clamp 1 (e.g. the number “4” or letter “D” or fourth other indicia). Therefore, if the roller clamp 1 does unexpectedly become dislodged by a violent movement and the roller wheel moves, then the operator can very quickly return the roller to the desired position because he or she has made a note of the position of the wheel.

The infusion rate scale and positioning scale can both be provided either printed directly onto the body of the roller clamp 1 or they could be applied on printed stickers. Alternatively, the scales 15 can be formed in the body of the roller clamp 1 for example where the roller clamp 1 can be manufactured from plastic and the scale can be provided during the moulding or curing process as a raised or indented area.

Alternatively, an infusion rate scale in millilitres per hour could be provided on both sides of the body of the roller clamp 1. Yet a further alternative would be to have the positioning scale with arbitrary units or indicia on both sides of the roller clamp 1.

When used in this specification and claims, the terms “comprises” and “comprising” and variations thereof mean that the specified features, steps or integers are included. The terms are not to be interpreted to exclude the presence of other features, steps or components.

The foregoing description of embodiments and examples has been presented for purposes of illustration and description. It is not intended to be exhaustive or limiting to the forms described. Numerous modifications are possible in light of the above teachings. Some of those modifications have been discussed, and others will be understood by those skilled in the art. The embodiments were chosen and described in order to best illustrate principles of various embodiments as are suited to particular uses contemplated. The scope is, of course, not limited to the examples set forth herein, but can be employed in any number of applications and equivalent devices by those of ordinary skill in the art.

1. A roller clamp comprising:
at least two opposing side walls spaced apart from each other and comprising respective interior surfaces;
a guide wall;
a further wall opposite the guide wall defining an aperture therethrough;
a hollow body having a body aperture therethrough, the body aperture being dimensioned and configured to house a resilient connection tube of a gravity infusion set therethrough, the hollow body defined by the at least two opposing and spaced apart side walls, the guide wall and the further wall opposite the guide wall;
a roller comprising a roller wheel having two axial projections protruding from a center of each side of the roller wheel, wherein:
the roller wheel protrudes through the aperture of the further wall;
the spacing between the guide wall and the further wall decreases over the length of the guide wall and the further wall;
each side wall is provided with an opposing guide groove on the interior surface thereof, each guide groove comprising at least one respective long edge;
the two axial projections of the roller wheel are slidingly seated in the guide grooves so that the roller can move in the aperture of the further wall as the projections slide in the guide grooves;
the at least one respective long edge of each corresponding guide groove is provided with at least one notch; and
the resulting at least one pair of corresponding notches can at least partially seat the two axial projections.

2. A roller clamp according to claim 1 wherein, in use, when a connection tube is housed in the hollow body, sliding of the roller in the direction from greater spacing to lesser spacing between the guide wall and the further wall causes the wheel to impinge on the connector tube to a gradually increasing extent, and wherein the axial projections become seated in the notches by return impinging force of the resilient tube.

3. A roller clamp according to claim 1 further comprising a plurality of corresponding pairs of notches which are arranged continuously in a serrated arrangement.

4. A roller clamp according to claim 1 wherein each at least one notch is provided in at least one of the following arrangements: closest to the further wall aperture; furthest from the further wall aperture; and within both long edges.

5. A roller clamp according to any preceding claim 1 wherein the guide wall further comprises a channel which has a depth which decreases in the same direction that the spacing between the guide wall and the further wall decreases.

6. A roller clamp according to claim 1 wherein the two axial projections are at least one of: formed from an axle passing through the roller wheel, fixedly attached to the roller wheel, and formed together with the roller wheel.

7. A roller clamp according to claim 1 further comprising indicia on the external surface of at least one side wall, wherein the indicia correspond to a position of the roller over the length of the hollow body.

8. A roller clamp according to claim 7 wherein the indicia are at least one of: arbitrary units including integers or alphanumeric characters, are markings including lines or dots, and a graduated scale such as a scale of infusion rate.

9. A roller clamp according to claim 8 wherein the indicia are a graduated scale of infusion rate.

10. A roller clamp according to claim 9 wherein the indicia are provided on the external surface of at least one side wall by at least one of: printing of the indicia directly on the at least one side wall, printing of the indicia on a sticker which is attached to the side wall or walls, and forming of the indicia directly in the constructional material of the at least one side wall.

11. A roller clamp according to claim 1 further comprising a plurality of notches which are formed in a continuous serrated arrangement along at least a portion of the guide grooves.

12. A gravity infusion set comprising the roller clamp of claim 1.

13. A gravity infusion set according to claim 12 wherein the gravity infusion set comprises a piercing means, a drop chamber, a connection tube and a connector; wherein:
the drop chamber comprises a drop generator;
the connector comprises a Luer connector; and
the connection tube is inserted through the body aperture.

14. A method of regulating fluid infusion through a connection tube, the method comprising:
inserting the connection tube through the body aperture of a roller clamp of claim 1; and
rolling the roller downwardly in the direction of greater to lesser spacing between the guide wall and the further wall, until the axial projections are seated in the at least one notch of each guide groove whereby a diameter of the connection tube is constricted to provide a desired flow rate of fluid through the connection tube.

15. A method according to claim 14, further comprising:
a user identifying indicia which are indicative of a desired flow rate on the at least one side wall.

16. (canceled)

17. (canceled)

18. A roller clamp according to claim 1 further comprising a plurality of corresponding pairs of notches which are spaced apart by un-notched regions of each respective guide groove.

19. A roller clamp according to claim 9 wherein the infusion rate is in units of ml per hour.
